(19) United States
(12) Patent Application Publication
(10) Pub. No.: US 2024/0209285 A1
(43) Pub. Date: Jun. 27, 2024

(54) ETHER SULFATES BASED ON ISOMERIC TRIDECYL ALCOHOL MIXTURES

(71) Applicant: BASF SE, Ludwigshafen am Rhein (DE)

(72) Inventors: Holger TUERK, Ludwigshafen (DE); Ozgecan ÖZEREN, Heidelberg (DE); Ditmar KISCHKEL, Düsseldorf-Holthausen (DE); Hans-Christian RATHS, Düsseldorf-Holthausen (DE)

(21) Appl. No.: 18/556,525

(22) PCT Filed: May 4, 2022
(86) PCT No.: PCT/EP22/62028

§ 371 Date: Oct. 20, 2023

(30) Foreign Application Priority Data
May 10, 2021 (EP) 21173099.9

(51) Int. Cl. C11D 1/29; C07C 315/02; C07C 317/18; C11D 1/37; C11D 3/00; C11D 3/34

(52) U.S. Cl. CPC C11D 1/29; C07C 315/02; C07C 317/18; C11D 1/37; C11D 3/0094; C11D 3/3454; C11D 2111/14

(57) ABSTRACT

Disclosed herein are a process of manufacturing ether sulfates based on isomeric tridecyl alcohol mixtures and ether sulfates based on isomeric tridecyl alcohol mixtures obtained or obtainable by such a process. Further disclosed herein are a composition including the ether sulfates based on isomeric tridecyl alcohol mixtures and a method of using the ether sulfates.

The present invention relates to ether sulfates based on isomeric tridecyl alcohol mixtures, compositions comprising the inventive ether sulfates based on isomeric tridecyl alcohol mixtures and their uses, in particular in detergent applications.

An important target of the detergent and cleaning industry is the improvement of the sustainability of detergent formulations, especially of liquid detergent formulations, leading to an increasing number of concentrated products on the market. Those concentrated products themselves contain a significantly lower amount of water. In addition, the amount of active ingredients (e.g., surfactants, builders, polymers, enzymes) per wash load is typically lower compared to the use of standard detergents, often due to compatibility issues.

Thus, there is a huge market need for more weight-efficient ingredients, especially in terms of cleaning performance. Since cleaning performance is often linked to foam appearance by end-consumers, there was an additional market need for materials that deliver an improved foam profile, e.g., under high soil load or at ultra-low surfactant concentrations.

Furthermore, there is also a need in the market for ingredients that are readily biodegradable.

EP 2264136 B1 and EP 2264138 B1 disclose the use of certain branched non-ionic surfactants, based on iso-tridecylalcohol (e.g., Lutensol TO7 from BASF), in combination with differently (i.e., lower) branched anionic surfactants, e.g., based on a C10 Guerbet alcohol, and furthermore in combination with specific chelating agents, in liquid hand dishwash formulations. The described dishwash formulations exhibit superior cleaning in combination with excellent shine properties. In addition, the described formulations also show good foaming properties, which could be furthermore improved by incorporation of a polymeric foam stabilizer. Hence, the overall properties of the final detergent formulation are tailormade by using several (three or more) different raw materials, which add a lot of complexity and cost to the product. Besides that, the polymeric foam stabilizer is typically non-biodegradable.

EP 1131400 B1 discloses surfactant granules containing alkyl ether sulfates based on linear or branched alcohols, including tridecylalcohol. The described granules have a high active content of up to 90 wt %. However, any application of the surfactant granules, e.g., in laundry detergents and/or dishwashing detergents, and any technical benefit of the ether sulfates besides their high active content is not mentioned. Furthermore, the use of tridecylalcohol as basis for preparation of the ether sulfates is not preferred.

EP 1465584 B1 discloses personal care formulations containing a branched ether sulfate (Rhodapex EST30, Solvay) based on a specific iso-tridecylalcohol. The specific iso-tridecylalcohol used for preparation of the inventive ether sulfate is prepared by oligomerization of propene and subsequent hydroformylation/hydrogenation, has a characteristically branched architecture with a degree of branching >3 and is always obtained as a mixture including many other C₉ to C₁₅ alcohols (Exxal 13, Exxon Mobil). The iso-tridecylalcohol content in the mixture is ca. 70 wt %. The branched anionic surfactant is then combined with cationic surfactants to obtain free-flowing personal care compositions with good stability at low temperatures. A positive contribution of the branched ether sulfates on the foam profile is mentioned, however, any laundry or dishwashing detergent formulation is not described at all. Furthermore, no information is given on the biodegradability of the branched ether sulfate.

The ingredients and formulations described in the prior art still have some shortcomings. For example, the detergent formulations disclosed in the prior art usually do not show good cleaning performance and good foam creation properties in combination. Besides, the disclosed ingredients are mostly not sufficiently biodegradable.

Thus, there was still a need to find improved ingredients for use in detergent compositions, preferably in detergent compositions that are already widely used in the market (i.e., that do not contain any cationic surfactants). In particular there was still a need to find a novel weight-efficient ingredient, preferably an anionic surfactant, that combines the properties of improved cleaning performance and superior foam creation and is readily biodegradable (>60%, OECD 301 B).

It was therefore an object of the present invention to provide improved ingredients, in particular anionic surfactants, for use in detergent compositions (as described above). In addition, it was an object to provide improved ingredients which can decrease the viscosity of a detergent composition, in order to improve its processability and its convenience for the end-consumer.

The inventors had the idea to try pure ether sulfates based on isomeric tridecyl alcohol mixtures for improved foam creation and improved cleaning properties in detergent & cleaners (D& C) applications.

The term “pure” in the context of the present invention is related to the alcohol component of the inventive alkyl ether sulfates and means single alcohol compounds or isomeric mixtures of alcohols having only one specific number of carbon atoms. Therefore, the term “pure ether sulfates based on isomeric tridecyl alcohol mixtures” in the context of the present invention means ether sulfates based on alcohols before ethoxylation and sulfation that solely contain thirteen carbon atoms. Consequently, many other iso-tridecylalcohol mixtures, e.g., the ones obtained by oligomerization of propene and subsequent hydroformylation/hydrogenation, leading to a C-chain distribution between C₉ and C₁₅ (Exxal 13, Exxon Mobil), are not falling under the term “pure” used in the present invention.

In the following, the term “ether sulfates based on isomeric tridecyl alcohol mixtures” is often used instead of the term “pure ether sulfates based on isomeric tridecyl alcohol mixtures” to describe the inventive materials of the present invention.

U.S. Pat. No. 6,963,014 describes the preparation of a pure and branched isomeric tridecylalcohol mixture and its derivatives. Alkoxylation and potentially subsequent sulfation of the obtained pure C₁₃ alcohol mixtures are mentioned only cursorily. Compositions containing, e. g., water in addition to the respective products are not disclosed or mentioned at all.

Surprisingly, it has now been found that the use of certain branched ether sulfates, based on an isomeric mixture of iso-tridecylalcohols (i.e., of alcohols with an identical number of thirteen carbon atoms, but different branching) and prepared in analogy to the process described in U.S. Pat. No. 6,963,014 (involving, inter alia, ethoxylation), leads to detergent formulations with improved cleaning properties and superior foam profile, especially in combination with other surfactants such as linear ether sulfates and/or linear alkylbenzene sulfonate. The benefit of the inventive branched ether sulfates is predominantly visible in manual hand dish application, where the novel ingredients lead to end-consumer perceivable benefits such as higher number of plates per wash (i.e., higher foam levels) and less manual effort in the cleaning process (i.e., better degreasing properties). In addition, the viscosity of highly concentrated formulations could be decreased which facilitates their preparation and use. Furthermore, the novel inventive ingredients are readily biodegradable.

Thus, one subject of the present invention are ether sulfates based on isomeric tridecyl alcohol mixtures, obtained by
a) bringing a butene-carrying C₄-hydrocarbon stream containing less than 5% by weight, based on the butene fraction, of isobutene into contact with a nickel-containing heterogeneous catalyst at elevated temperature,
b)isolating a C₁₂-olefin fraction from the reaction mixture,
c) hydroformylating the C₁₂-olefin fraction by reaction with carbon monoxide and hydrogen in the presence of a cobalt catalyst,
d)hydrogenating the product from c),
e)ethoxylating the product from d), wherein 1 to 5 EO units per OH group are used on average,
f) sulfating the product from e) with SO₃, preferably in a falling-film reactor, and
g) neutralizing the product from f) with an aqueous solution of a base, preferably selected from the group consisting of alkaline metals, earth alkaline metals and amines.

Thus, the inventive ether sulfates are based on ethoxylated iso-tridecylalcohols only, and not on e.g., propoxylated or mixed (e.g., EO/PO) iso-tridecylalcohols.

In a preferred embodiment of the inventive ether sulfates based on isomeric tridecyl alcohol mixtures, the C₁₂-olefin fraction has an ISO index of from 1.9 to 2.3.

In another preferred embodiment of the inventive ether sulfates based on isomeric tridecyl alcohol mixtures, the butene-carrying C₄-hydrocarbon stream comprises from 60 to 90% by weight of butenes and from 10 to 40% by weight of butanes.

In another preferred embodiment of the inventive ether sulfates based on isomeric tridecyl alcohol mixtures, the nickel-containing heterogeneous catalyst comprises nickel oxide.

In another preferred embodiment of the inventive ether sulfates based on isomeric tridecyl alcohol mixtures es, the nickel-containing heterogeneous catalyst consists essentially of NiO, SiO₂, TiO₂ and/or ZrO₂ and, if desired, Al₂O₃.

In a further preferred embodiment of the inventive ether sulfates based on isomeric tridecyl alcohol mixtures, 2 to 4EO units per OH group are used on average in step e), preferably 2.5 to 3.5, more preferably about 3.

In a further preferred embodiment of the inventive ether sulfates based on isomeric tridecyl alcohol mixtures, the sulfation in step f) is done in a falling film sulfonation reactor, preferably at a reactor temperature of 10° to 60° C., preferably 20° to 50° C., and/or with a molar ratio of SO₃/alcohol ethoxylate of 0.9 to 1.20, preferably 0.95 to 1.15.

In a further preferred embodiment of the inventive ether sulfates based on isomeric tridecyl alcohol mixtures, the degree of sulfation in step f) is from 80% to 100%, preferably 90% to 100%, more preferably from 93% to 100% and most preferred from 95% to 99%.

In a further preferred embodiment of the inventive ether sulfates based on isomeric tridecyl alcohol mixtures, the neutralization in step g) is performed with an aqueous solution of a base selected from the list consisting of NaOH, KOH, triethanolamine, ammonia, preferably NaOH.

In a further preferred embodiment of the inventive ether sulfates based on isomeric tridecyl alcohol mixtures, the iso-tridecylalcohol obtained after steps a) to d) above has a mean degree of branching of from 2.1 to 2.5, preferably from 2.2 to 2.4.

The mean degree of branching is defined as the number of methyl groups in one molecule of the ether sulfate minus 1.

The mean degree of branching is the statistical mean of the degree of branching of the molecules of a sample. The mean number of methyl groups in the molecules of a sample can easily be determined by 1H-NMR spectroscopy. For this purpose, the signal area corresponding to the methyl protons in the 1H-NMR spectrum of a sample is divided by three and then divided by the signal area of the methylene protons of the CH₂—OH group divided by two.

Comparable products known in the art, specifically branched ether sulfates based on iso-tridecylalcohol prepared by oligomerization of propene and subsequent hydroformylation/hydrogenation, commercially available as Exxal 13 (Exxon Mobil), usually have a higher degree of branching than the inventive ether sulfates, for example a mean degree of branching of greater than 2.5. Furthermore, they are not pure C₁₃ based compounds, since the alcohol component used for their preparation is always based on a mixture including many other C₉ to C₁₅ alcohols.

In a further preferred embodiment, the inventive ether sulfates based on isomeric tridecyl alcohol mixtures have a biodegradability according to OECD norm 301B (measuring of CO₂ evolution, respirometric method) of at least 60% (after 28 days) and are thus considered to be readily biodegradable.

A measured volume of inoculated mineral medium, containing a known concentration of the test substance (10-20 mg DOC or TOC/I) as the nominal sole source of organic carbon is aerated by the passage of carbon dioxide-free air at a controlled rate in the dark or in diffuse light. Degradation is followed over 28 days by determining the carbon dioxide produced. The CO₂ is trapped in barium or sodium hydroxide and is measured by titration of the residual hydroxide or as inorganic carbon. The amount of carbon dioxide produced from the test substance (corrected for that derived from the blank inoculum) is expressed as a percentage of ThCO₂. The degree of biodegradation may also be calculated from supplemental DOC analysis made at the beginning and end of incubation.

Comparable products known in the art, specifically branched ether sulfates based on isomeric tridecyl alcohol mixtures prepared by oligomerization of propene and subsequent hydroformylation/hydrogenation, commercially available as Exxal 13 (Exxon Mobil), are usually not readily biodegradable, i.e. have a biodegradability according to OECD norm 301B of less than 60% (after 28 days). These products do not fulfill the market needs for sustainable products in the home care and detergent industry.

The inventive ether sulfates based on isomeric tridecyl alcohol mixtures usually are in the form of a sticky solid, which may cause problems in some production plants. Thus, the inventors had the idea to add water, such that a lower viscosity can be obtained in order to make the product more easily processable.

Thus, another subject of the present invention is also a composition comprising inventive ether sulfates based on isomeric tridecyl alcohol mixtures (as described above) and water.

Usually, the inventive composition contains 5 to 99% by weight water.

In a preferred embodiment of the present invention, the composition comprising inventive ether sulfates based on isomeric tridecyl alcohol mixtures (as described above) and water contains preferably 5 to 50% by weight, more preferably 10 to 50 weight %, even more preferably 20 to 40 weight %, most preferably 25 to 35 weight % of water. Typically, the inventive composition with 5 to 50% by weight of water is available as a paste with a processable viscosity (<15 Pa*s, preferably <10 Pa*s; at 20° C. and shear rate 10 1/s, on a rotational rheometer and plate-plate geometry), especially at temperatures of 20 to 40° C.

In another preferred embodiment of the present invention, the composition comprising inventive ether sulfates based on isomeric tridecyl alcohol mixtures (as described above) and water contains preferably 50 to 99% by weight, more preferably 70 to 95 weight %, even more preferably 75 to 90 weight %, most preferably 80 to 90 weight % of water. Typically, the inventive composition with 50 to 99% by weight of water is available as a free-flowing liquid with low or even very low viscosity (<10 Pa*s, preferably <1 Pa*s; at 20° C. and shear rate 10 1/s, on a rotational rheometer and plate-plate geometry), also suitable for direct use in cleaning applications.

A further subject of the present invention is also a composition, preferably liquid composition, as described above, comprising additionally at least one anionic surfactant, selected from the group consisting of other ether sulfates, alkyl sulfates and LAS.

In an embodiment of the invention, the composition further comprises at least one co-surfactant, preferably selected from the group consisting of amine oxides, betaines, alkyl sulfates, ester sulfonates, alkyl polyglucosides and non-ionic surfactants based on C₈ to C₁₀ alcohol alkoxylates, more preferably selected from the group consisting of amine oxides, betaines and ethoxylated C₁₀ Guerbet alcohol surfactants with a number average degree of ethoxylation in the range of 2.5 to 8, most preferably selected from amine oxides and betaines.

In another embodiment of the invention, the composition does not contain any cationic surfactants.

In another embodiment of the invention, the composition does not contain any non-ionic surfactants based on C₁₂ to C₂₀ alcohol alkoxylates, preferably does not contain any non-ionic surfactants based on C₁₂ to C₁₈ alcohol alkoxylates.

In another embodiment of the invention, the composition contains up to 70 wt %, preferably up to 50 wt %, more preferably up to 30 wt % ether sulfates based on isomeric tridecyl alcohol mixtures (relating to the active matter content comprising or consisting of ether sulfates based on isomeric tridecyl alcohol mixtures, other anionic surfactants and co-surfactants). In one embodiment, the composition contains at least 5 wt %, preferably at least 10 wt % ether sulfates based on isomeric tridecyl alcohol mixtures (relative to the active matter content comprising or consisting of ether sulfates based on isomeric tridecyl alcohol mixtures, other anionic surfactants and co-surfactants).

In another embodiment of the invention, the composition contains 30 to 95 wt % of an additional anionic surfactant, selected from the group consisting of other ether sulfates and LAS (linear alkylbenzene sulfonate), preferably from 50 to 95 wt %, more preferably from 60 to 95 wt % and most preferably from 70 to 90 wt %, relative to active matter comprising or consisting of ether sulfates based on isomeric tridecyl alcohol mixtures, other ether sulfates, alkyl sulfates and LAS.

Nonlimiting examples of alkyl sulfates—which may be employed also in combinations of more than one surfactant—useful herein include C10-C20 primary, branched chain and random alkyl sulfates; C10-C18 secondary (2,3) alkyl sulfates; and mid-chain branched alkyl sulfates as discussed in U.S. Pat. Nos. 6,020,303 and 6,060,443. Preferred examples of suitable alkyl sulfates are alkali metal and ammonium salts of C8-C12-alkyl sulfates.

In a preferred embodiment of the invention, the composition is a detergent composition, preferably a dishwashing detergent composition or a hard surface cleaner detergent composition, more preferably a manual dishwashing detergent composition.

In another embodiment of the invention, the composition is a laundry detergent composition, more preferably a liquid laundry detergent composition.

A further subject of the present invention is the use of the inventive ether sulfates based on isomeric tridecyl alcohol mixtures, as described above, for increasing the foam level in a detergent composition, reducing the viscosity of a detergent composition and/or improving the degreasing properties of a detergent composition.

The foam level of a detergent composition may be measured in accordance with the plates wash test (see examples). An increase of the number of plates that can be washed is at least 2 plates.

The viscosity of a detergent composition may be determined by a rheometer, e.g., a rotational rheometer (Brookfield; See examples). The viscosity of the formulation may be decreased by at least 10%, preferably by 30%, more preferably by at least 50%.

The degreasing properties of a detergent composition can be measured as follows: Using the emulsification stability test (see examples).

In an embodiment of the present invention, the foam level of a detergent composition is increased considerably. For example, the foam level of a detergent composition is increased by at least 20 percent, preferably at least 30 percent by using the inventive compound. The increase of the foam level could be detected e.g., by measuring the foam volume or foam height in a given container after manual agitation (see examples).

In another embodiment of the present invention, the viscosity of a detergent composition is reduced considerably. For example, the viscosity of a detergent composition is reduced by at least 10 percent, preferably at least 30 percent, more preferably at least 50 percent by using the inventive compound, compared to a detergent composition with the same active matter content and based on linear or predominantly linear C₁₂-C₁₈-alcohol ether sulfates instead.

In a further embodiment of the present invention, the degreasing properties of the detergent composition are improved considerably. For example, the degreasing properties of the detergent composition are increased by at least 10 percent, preferably at least 20 percent, more preferably at least 30 percent by using the inventive compound, compared to a detergent composition with the same active matter content and based on linear or predominantly linear C₁₂-C₁₈-alcohol ether sulfates instead.

The inventive ether sulfates based on isomeric tridecyl alcohol mixtures may be prepared as follows.

### Isomeric Tridecyl Alcohol Mixture

The isomeric tridecylalcohol mixture used for preparing the inventive ether sulfates based on isomeric tridecyl alcohol mixtures may be manufactured as described in U.S. Pat. No. 6,963,014 B1.

### Ethoxylation

Ethoxylation of the isomeric tridecylalcohol mixture may be performed in accordance with the disclosures of U.S. Pat. No. 6,963,014 B1.

### Sulfation (Followed by Neutralization)

The alcohol ethoxylate from the previous step is usually kept in a stirred storage vessel, generally at a temperature in the range of 30° to 80° C., preferably 40° to 70° C., more preferably 50° to 60° C.

The sulfation is preferably done in a falling film reactor. The reactor temperature is usually in the range of 5° C. to 80° C., preferably 10° to 60° C., more preferably 20° to 50° C. For example, the reactor temperature may be around 30° C. or around 35° C. or around 45° C.

The molar ratio of SO₃/alcohol ethoxylate is usually in the range of 0.9 to 1.20, preferably 0.95 to 1.15. The molar ratio of SO₃/alcohol ethoxylate may, for example, be in the range of 1.0 to 1.05, with dry air/SO₃ containing 5 vol % of SO₃.

The degree of sulfation is from 80% to 100%, preferably 90% to 100%, even more preferably from 93% to 100% and most preferred from 95% to 99%.

Neutralization is usually performed after a degassing step. The neutralization may be carried out with NaOH, KOH and amines like TEA (triethanolamine). Preferred is NaOH, to yield the sodium salt of the ether sulfate. The neutralization may advantageously be performed with an aqueous solution of a base selected from the list consisting of NaOH, KOH, triethanolamine, ammonia, preferably NaOH.

The water content may be selected to obtain a concentration of active compound in the range of 1 to 50% by weight, more preferably 5 to 30 weight %, even more preferably 10 to 25 weight %, most preferably 10 to 20% by weight, at a temperature of 65° C.

The pH value is usually selected to be between 9 to 12, preferably 10 to 11, to avoid hydrolysis of the product.

In a preferred embodiment, the amount of water is reduced to obtain a 50 to 95% by weight, more preferably 50 to 90 weight %, even more preferably 60 to 80 weight %, most preferably 65 to 75 weight % active surfactant paste. In order to produce this high concentrated paste, it is beneficial to do the neutralization in a neutralization loop.

The resulting product can be bleached either during neutralization, which is beneficial for the 70% active paste, or in a stirred vessel, for example by applying 0.1-0.5% hydrogen peroxide as 35% solution at 50-80° C.

The final pH can be adjusted, for example by adding caustic soda or citric acid or sodium carbonate solution to the desired pH value.

The inventive ether sulfates based on isomeric tridecyl alcohol mixtures may be used in compositions containing water, for example detergent compositions. For a detergent composition, the term “detergent formulation” is sometimes also used synonymously.

In one embodiment of the present invention, the detergent compositions comprise at least one inventive ether sulfates based on isomeric tridecyl alcohol mixture and at least one additional anionic surfactant, selected from the group consisting of other ether sulfates, alkyl sulfates and LAS.

Liquid detergent compositions are preferred.

### (a) Manual Dish Wash Detergents

An aspect of the present invention is also a manual dish wash detergent formulation (manual dish wash detergent composition), comprising at least one of the inventive ether sulfates based on isomeric tridecyl alcohol mixtures as described above.

Thus, an aspect of the present invention is also the use of the inventive ether sulfates based on isomeric tridecyl alcohol mixtures, as described above, in manual dish wash applications.

Manual dish wash detergents according to the invention can be in the form of a liquid, semiliquid, cream, lotion, gel, or solid composition, solid embodiments encompassing, for example, powders and tablets. Liquid compositions are preferred. The detergents are intended for use in liquid form for direct or indirect application onto dishware.

In one embodiment of the present invention, the inventive ether sulfates based on isomeric tridecyl alcohol mixtures are one component of a manual dish wash formulation that additionally comprises at least one other surfactant, preferably at least one other anionic surfactant.

Preferably the composition is formulated for increasing the foam level of a detergent composition (i.e., increasing the number of plates that can be washed with the detergent composition), reducing the viscosity of a detergent composition and/or improving the degreasing properties of a detergent composition. Optional further benefits include delivery of long-lasting suds and improvement of soil removal, shine and hand care.

Examples of suitable additional anionic surfactants are alkali metal and ammonium salts of C₁₂-C₁₈-fatty alcohol ether sulfates, of C₁₂-C₁₈-fatty alcohol polyether sulfates, of sulfuric acid half-esters of ethoxylated C₄-C₁₂-alkylphenols (ethoxylation: 3 to 50 mol of ethylene oxide/mol), of C₁₂-C₁₈-alkylsulfonic acids, of C₁₀-C₁₈-alkylarylsulfonic acids, preferably of n-C₁₀-C₁₈-alkylbenzene sulfonic acids (LAS), of C₁₀-C₁₈ alkyl alkoxy carboxylates and of soaps such as for example C8-C24-carboxylic acids. Preference is given to the alkali metal salts of the aforementioned ingredients, particularly preferably the sodium salts.

Further suitable additional anionic surfactants are alkyl sulfates. Nonlimiting examples of alkyl sulfates—which may be employed also in combinations of more than one surfactant—useful herein include C10-C20 primary, branched chain and random alkyl sulfates; C10-C18 secondary (2,3) alkyl sulfates; and mid-chain branched alkyl sulfates as discussed in U.S. Pat. Nos. 6,020,303 and 6,060,443. Preferred examples of suitable alkyl sulfates are alkali metal and ammonium salts of C8-C12-alkyl sulfates.

In a preferred embodiment of the present invention, additional anionic surfactants are selected from n-C₁₀-C₁₈-alkylbenzene sulfonic acids (LAS), from fatty alcohol polyether sulfates, which, within the context of the present invention, are in particular sulfuric acid half-esters of ethoxylated C₁₂-C₁₈-alkanols (ethoxylation: 1 to 50 mol of ethylene oxide/mol), preferably of n-C₁₂-C₁₈-alkanols, and from C₈-C₁₂-alkyl sulfates.

In one embodiment of the present invention, also alcohol polyether sulfates derived from other synthetic C₁₁-C₁₈-alkanols (ethoxylation: 1 to 50 mol of ethylene oxide/mol) may be employed.

Preferably, the alkoxylation group of both types of alkoxylated alkyl sulfates, based on C₁₂-C₁₈-fatty alcohols or based on other synthetic C₁₁-C₁₈-alcohols, is an ethoxylation group and an average ethoxylation degree of any of the alkoxylated alkyl sulfates is 1 to 5, preferably 1 to 3.

Preferably, the manual dish wash detergent formulation of the present invention comprises from at least 30 wt % to 95 wt %, preferably in the range from greater than or equal to about 50 wt % to equal to or less than about 95 wt %, more preferably in the range from greater than or equal to 60 wt % to less than or equal to 95 wt %, and most preferably in the range from greater than or equal to 70 wt % to less than or equal to 90 wt % of one or more additional anionic surfactants, preferably selected from n-C₁₀-C₁₈-alkylbenzene sulfonic acids (LAS), from fatty and/or synthetic alcohol polyether sulfates and from C₈-C₁₂-alkyl sulfates, as described above, relative to active matter comprising ether sulfates based on isomeric tridecyl alcohol mixtures, other ether sulfates, alkyl sulfates and LAS, i.e. based on the particular overall composition but without other components, water and/or solvents.

In a preferred embodiment of the present invention, the manual dish wash detergent compositions based on the inventive ether sulfates based on isomeric tridecyl alcohol mixtures and the at least one other surfactant, preferably the at least one other anionic surfactant, can be obtained as clear, transparent and homogeneous liquid formulations at room temperature (25° C.), without any turbidity or precipitation of components.

In contrast, comparable products known in the art, specifically branched ether sulfates based on iso-tridecylalcohol prepared by oligomerization of propene and subsequent hydroformylation/hydrogenation, commercially available as Exxal 13 (Exxon Mobil), typically lead to cloudy, non-transparent and inhomogeneous liquid formulations at room temperature (25° C.), along the way often with precipitation of components, if combined with at least one other surfactant, preferably with at least one other anionic surfactant, more preferably if combined with LAS. Such non-transparent formulations are instable and thus technically not viable.

In one embodiment of the present invention, the inventive ether sulfates based on isomeric tridecyl alcohol mixtures are one component of a manual dish wash formulation that additionally comprises at least one other anionic surfactant as described above, and further comprising at least one additional co-surfactant, preferably selected from the group consisting of amine oxides, betaines, alkyl sulfates, ester sulfonates, alkyl polyglucosides and non-ionic surfactants based on C₈ to C₁₀ alcohol alkoxylates.

In another embodiment of the present invention, the inventive ether sulfates based on isomeric tridecyl alcohol mixtures are one component of a manual dish wash formulation that additionally comprises at least one additional co-surfactant, preferably selected from the group consisting of amine oxides, betaines, alkyl sulfates, ester sulfonates, alkyl polyglucosides and non-ionic surfactants based on C₈ to C₁₀ alcohol alkoxylates, and no further anionic surfactant as described above.

Hence, compositions according to the invention may comprise at least one amphoteric surfactant as co-surfactant.

Examples of amphoteric co-surfactants are amine oxides. Preferred amine oxides are alkyl dimethyl amine oxides or alkyl amido propyl dimethyl amine oxides, more preferably alkyl dimethyl amine oxides and especially coco dimethyl amino oxides. Amine oxides may have a linear or mid-branched alkyl moiety. Typical linear amine oxides include water-soluble amine oxides containing one R₁═C₈₋₁₈ alkyl moiety and two R₂ and R₃ moieties selected from the group consisting of C₁-C₃ alkyl groups and C₁-C₃ hydroxyalkyl groups. Preferably, the amine oxide is characterized by the formula

R₁—N(R₂)(R₃)—O

wherein R₁ is a C₈₋₁₈ alkyl and R₂ and R₅ are selected from the group consisting of methyl, ethyl, propyl, isopropyl, 2-hydroxethyl, 2-hydroxypropyl and 3-hydroxypropyl. The linear amine oxide co-surfactants in particular may include linear C₁₀-C₁₈ alkyl dimethyl amine oxides and linear C₈-C₁₂ alkoxy ethyl dihydroxy ethyl amine oxides. Preferred amine oxides include linear C₁₀, linear C₁₀-C₁₂, and linear C₁₂-C₁₄ alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the alpha carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from 10 to 16. The number of carbon atoms for the one alkyl moiety (n1) should be approximately the same number of carbon atoms as the one alkyl branch (n2) such that the one alkyl moiety and the one alkyl branch are symmetric. As used herein “symmetric” means that (n1-n2) is less than or equal to 5, preferably 4, most preferably from 0 to 4 carbon atoms in at least 50 wt %, more preferably at least 75 wt % to 100 wt % of the mid-branched amine oxides for use herein. The amine oxide further comprises two moieties, independently selected from a C₁-C₃ alkyl, a C₁-C₃ hydroxyalkyl group, or a polyethylene oxide group containing an average of from about 1 to about 3 ethylene oxide groups. Preferably the two moieties are selected from a C₁-C₃ alkyl, more preferably both are selected as a C₁ alkyl.

The manual dish wash detergent composition of the invention optionally comprises from 1 wt % to 15 wt %, preferably from 2 wt % to 12 wt %, more preferably from 3 wt % to 10 wt % of the composition including all components, water and solvents, of an amphoteric co-surfactant, preferably an amine oxide co-surfactant. Preferably the composition of the invention comprises a mixture of the anionic surfactants, based on the inventive ether sulfates based on isomeric tridecyl alcohol mixtures, other ether sulfates, alkyl sulfates and LAS, and alkyl dimethyl amine oxides in a weight ratio of less than about 10:1, more preferably less than about 8:1, more preferably from about 5:1 to about 2:1.

Addition of the amphoteric co-surfactant may provide good foaming properties in the detergent composition.

Compositions according to the invention may comprise at least one zwitterionic co-surfactant. Suitable zwitterionic co-surfactants include betaines, such as alkyl betaines, alkylamidobetaine, amidazoliniumbetaine, sulfobetaine (INCI Sultaines) as well as the phosphobetaines. Examples of suitable betaines and sulfobetaines are the following (designated in accordance with INCI): Almondamidopropyl of betaines, Apricotam idopropyl betaines, Avocadamidopropyl of betaines, Babassuamidopropyl of betaines, Behenam idopropyl betaines, Behenyl of betaines, Canolam idopropyl betaines, Capryl/Capram idopropyl betaines, Carnitine, Cetyl of betaines, Cocamidoethyl of betaines, Cocam idopropyl betaines, Cocam idopropyl Hydroxysultaine, Coco betaines, Coco Hydroxysultaine, Coco/Oleam idopropyl betaines, Coco Sultaine, Decyl of betaines, Dihydroxyethyl Oleyl Glycinate, Dihydroxyethyl Soy Glycinate, Dihydroxyethyl Stearyl Glycinate, Dihydroxyethyl Tallow Glycinate, Dimethicone Propyl of PG-betaines, Erucam idopropyl Hydroxysultaine, Hydrogenated Tallow of betaines, Isostearam idopropyl betaines, Lauram idopropyl betaines, Lauryl of betaines, Lauryl Hydroxysultaine, Lauryl Sultaine, Milkam idopropyl betaines, Minkamidopropyl of betaines, Myristam idopropyl betaines, Myristyl of betaines, Oleam idopropyl betaines, Oleam idopropyl Hydroxysultaine, Oleyl of betaines, Olivamidopropyl of betaines, Palmam idopropyl betaines, Palm itam idopropyl betaines, Palmitoyl Carnitine, Palm Kernelam idopropyl betaines, Polytetrafluoroethylene Acetoxypropyl of betaines, Ricinoleam idopropyl betaines, Sesam idopropyl betaines, Soyam idopropyl betaines, Stearam idopropyl betaines, Stearyl of betaines, Tallowam idopropyl betaines, Tallowam idopropyl Hydroxysultaine, Tallow of betaines, Tallow Dihydroxyethyl of betaines, Undecylenam idopropyl betaines and Wheat Germam idopropyl betaines.

A preferred betaine is, for example, Cocoamidopropylbetaine. The zwitterionic co-surfactant preferably is a betaine surfactant, more preferable a Cocoamidopropylbetaine co-surfactant.

The manual dish wash detergent composition of the invention optionally comprises from 0.5 wt % to 15 wt %, preferably from 1 wt % to 12 wt %, more preferably from 2 wt % to 10 wt % of the composition including all components, water and solvents, of a zwitterionic co-surfactant, preferably a betaine co-surfactant.

Compositions according to the invention may comprise at least one additional anionic co-surfactant.

Examples of suitable additional anionic co-surfactants are alkali metal and ammonium salts of of C₈-C₁₆-alkyl sulfates and of C₁₂-C₁₈ sulfo fatty acid alkyl esters, for example of C₁₂-C₁₈ sulfo fatty acid methyl esters.

The manual dish wash detergent composition of the invention optionally comprises from 0.5 wt % to 10 wt %, preferably from 1 wt % to 8 wt %, more preferably from 2 wt % to 5 wt % of the composition including all components, water and solvents, of an additional anionic co-surfactant, preferably an alkali metal salt of C₈-C₁₆-alkyl sulfates.

Formulations according to the invention may also contain at least one alkyl polyglucoside as cosurfactant.

Alkyl polyglycosides and methods for their manufacture are known per se.

In one embodiment of the present invention, the alkyl polyglycoside is selected from those of general formula (I)

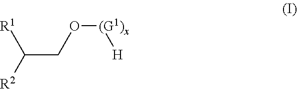

wherein the integers are defined as follows:
R¹ is selected from C₁-C₄-alkyl, branched or preferably linear, for example methyl, ethyl, n-propyl, n-butyl, or preferably hydrogen,
R² is C₃-C₁₂-alkyl, branched or preferably linear, for example n-propyl, n-butyl, iso-butyl, n-pentyl, iso-amyl, n-hexyl, n-heptyl, iso-heptyl, n-octyl, iso-octyl, n-decyl or n-dodecyl.
G¹ selected from monosaccharides with 4 to 6 carbon atoms,
x in the range of from 1.1 to 3.

In one embodiment of the present invention, R¹ and R² are selected independently from each other.

In a preferred embodiment of the present invention, R¹ and R² are selected interdependently from each other.

For example, if R¹ is selected from ethyl, then R² is selected from n-butyl. In a further example R¹ is selected from C₃-alkyl, linear or branched and R² is selected from C₅-alkyl, linear or branched. In a further example R¹ is selected from C₄-alkyl, linear or branched, and R² is selected from C₆-alkyl, linear or branched.

In a preferred embodiment of the present invention, R¹ is selected from hydrogen and R² is selected from linear C₆-C₁₀-alkyl.

In another particularly preferred embodiment of the present invention, R¹ is n-C₃H₇ and R² is n-C₅H₁₁.

G¹ is selected from monosaccharides, preferably from tetroses, pentoses, and hexoses. Examples of tetroses are erythrose, threose, and erythulose. Examples of pentoses are ribulose, xylulose, ribose, arabinose, xylose and lyxose. Examples of hexoses are galactose, mannose and glucose. Monosaccharides may be synthetic or derived or isolated from natural products, hereinafter in brief referred to as natural saccharides, and natural saccharides being preferred. More preferred are the following natural monosaccharides: galactose, xylose, and in particular glucose. Monosaccharides can be selected from any of their enantiomers, naturally occurring enantiomers and naturally occurring mixtures of enantiomers being preferred.

The integer x is a number in the range of from 1.1 to 3, preferred are 1.1 to 2 and in particularly preferred are 1.15 to 1.9. In the context of the present invention, x refers to an average value, and x is not necessarily a whole number.

In a specific alkyl polyglucoside, only whole groups of G¹ can occur. In single molecules of the alkyl polyglucoside there may be, for example, only one G¹ moiety or up to 15 G¹ moieties per molecule.

In single molecules of the alkyl polyglucoside with 2 or more G¹ moieties, the monosaccharide groups (sugar molecules) can be linked in any position, for example, in 1,6-position, in 1,2-position or in 1,3-position and preferably in 1,6-position or 1,4-position. The linkage can be α or β.

The manual dish wash detergent composition of the invention optionally comprises from 0.5 wt % to 15 wt %, preferably from 1 wt % to 12 wt %, more preferably from 2 wt % to 10 wt % of the composition including all components, water and solvents, of an alkyl polyglucoside as a cosurfactant.

Compositions according to the invention may comprise at least one non-ionic surfactant.

Preferred non-ionic surfactants are alkoxylated alcohols and alkoxylated fatty alcohols, di- and multiblock copolymers of ethylene oxide and propylene oxide and reaction products of sorbitan with ethylene oxide or propylene oxide, furthermore alkylphenol ethoxylates, alkyl glycosides and polyhydroxy fatty acid amides (glucamides).

Preferred examples of alkoxylated alcohols and alkoxylated fatty alcohols are, for example, ingredients of the general formula (II)

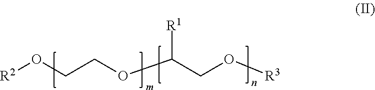

in which the variables are defined as follows:
R¹ is selected from linear C₁-C₁₀-alkyl, preferably ethyl and particularly preferably methyl,
R² is selected from C₈-C₂₂-alkyl, for example n-C₈H₁₇, n-C₁₀H₂₁, n-C₁₂H₂₅, n-C₁₄H₂₉, n-C₁₆H₃₃ or n-C₁₈H₃₇,
R³ is selected from hydrogen or C₁-C₁₀-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl,

m and n are in the range from zero to 300, where the sum of n and m is at least one. Preferably, m is in the range from 1 to 100 and n is in the range from 0 to 30.

Here, ingredients of the general formula (II) may be block copolymers or random copolymers, preference being given to block copolymers.

Other preferred examples of alkoxylated alcohols and alkoxylated fatty alcohols are, for example, ingredients of the general formula (III)

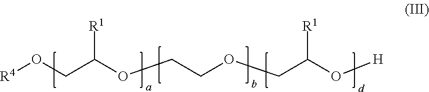

in which the variables are defined as follows:
R¹ is identical or different and selected from linear C₁-C₄-alkyl, preferably identical in each case and ethyl and particularly preferably methyl,
R⁴ is selected from C₆-C₂₀-alkyl, in particular n-C₈H₁₇, n-C₁₀H₂₁, n-C₁₂H₂₅, n-C₁₄H₂₉, n-C₁₆H₃₃, n-C₁₈H₃₇,
a is a number in the range from zero to 6, preferably 1 to 6,
b is a number in the range from zero to 20, preferably 4 to 20,
d is a number in the range from 4 to 25.

Preferably, at least one of a and b is greater than zero.

Here, ingredients of the general formula (III) may be block copolymers or random copolymers, preference being given to block copolymers.

Further suitable non-ionic surfactants are selected from di- and multiblock copolymers, composed of ethylene oxide and propylene oxide. Further suitable non-ionic surfactants are selected from ethoxylated or propoxylated sorbitan esters. Alkylphenol ethoxylates or alkyl polyglycosides or polyhydroxy fatty acid amides (glucamides) are likewise suitable. An overview of suitable further non-ionic surfactants can be found in EP-A 0 851 023 and in DE-A 198 19 187.

Mixtures of two or more different non-ionic surfactants may also be present.

Preferred non-ionic surfactants that are used in the inventive formulations as co-surfactants are ingredients of the general formula (IV)

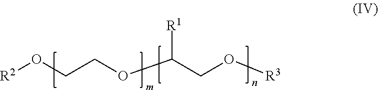

in which the variables are defined as follows:
R¹ is methyl,
R² is selected from C₈-C₁₀-alkyl, linear or branched, more preferably 2-propylheptyl
R³ is hydrogen

m is in the range from 1 to 20, n is in the range of zero to 5, where the sum of n and m is at least one. More preferably, m is in the range from 2.5 to 8 and n is zero.

Thus, most preferred non-ionic surfactants that are used in the inventive formulations as cosurfactants are the condensation products of 2-propylheptanol (Guerbet alcohol) with from 2.5 to 8 moles, more preferably 2.5 to 6, even more preferably 3 to 5 of ethylene oxide per mole of alcohol.

The manual hand dish detergent composition of the present invention optionally comprises from 0.1 wt % to 10 wt %, preferably from 0.3 wt % to 5 wt %, more preferably from 0.4 wt % to 2 wt % of the composition including all components, water and solvents, of a linear or branched C₈-C₁₀ alkoxylated non-ionic co-surfactant having an average degree of alkoxylation of from 1 to 25, preferably from 2.5 to 8. Preferably, the linear or branched C₁₀ alkoxylated non-ionic cosurfactant is a branched C₁₀ ethoxylated non-ionic co-surfactant having an average degree of ethoxylation of from 2.5 to 8, preferably of from 2.5 to 6, more preferably from 3 to 5.

Preferably, the composition comprises from 60 wt % to 100 wt %, preferably from 80 wt % to 100 wt %, more preferably 100 wt % of the total linear or branched C₁₀ alkoxylated non-ionic cosurfactant of the branched C₁₀ ethoxylated non-ionic co-surfactant. The linear or branched C₁₀ alkoxylated non-ionic co-surfactant preferably is a 2-propylheptyl ethoxylated non-ionic cosurfactant having an average degree of ethoxylation of from 3 to 5. A suitable 2-propylheptyl ethoxylated non-ionic co-surfactant having an average degree of ethoxylation of 4 is Lutensol® XP40, commercially available from BASF SE, Ludwigshafen, Germany. The use of a 2-propylheptyl ethoxylated non-ionic co-surfactant having an average degree of ethoxylation of from 3 to 5 leads to further improved foam levels and long-lasting suds.

Compositions according to the invention may comprise at least one hydrotrope in an effective amount, to ensure the compatibility of the liquid manual dish wash detergent compositions with water.

Suitable hydrotropes for use herein include anionic hydrotropes, particularly sodium, potassium, and ammonium xylene sulfonate, sodium, potassium and ammonium toluene sulfonate, sodium, potassium, and ammonium cumene sulfonate, and mixtures thereof, and related ingredients, as disclosed in U.S. Pat. No. 3,915,903.

The liquid manual dish wash detergent compositions of the present invention optionally comprise from 0.1 wt % to 15 wt % of the total liquid detergent composition of a hydrotrope, or mixtures thereof, preferably from 1 wt % to 10 wt %, most preferably from 2 wt % to 5 wt % of the total liquid manual dish wash composition including all components, water and solvents.

Compositions according to the invention may comprise at least one organic solvent.

Examples of organic solvents are C₄-C₁₄ ethers and diethers, glycols, alkoxylated glycols, C₆-C₁₆ glycol ethers, alkoxylated aromatic alcohols, aromatic alcohols, aliphatic branched alcohols, alkoxylated aliphatic branched alcohols, alkoxylated linear C₁-C₅ alcohols, linear C₁-C₅ alcohols, amines, C₈-C₁₄ alkyl and cycloalkyl hydrocarbons and halohydrocarbons, and mixtures thereof. When present, the liquid detergent compositions will contain from 0.01 wt % to 20 wt %, preferably from 0.5 wt % to 15 wt %, more preferably from 1 wt % to 10 wt %, most preferably from 1 wt % to 5 wt % of the liquid detergent composition of a solvent. These solvents may be used in conjunction with an aqueous liquid carrier, such as water, or they may be used without any aqueous liquid carrier being present. At higher solvent systems, the absolute values of the viscosity may drop but there is a local maximum point in the viscosity profile.

In a preferred embodiment of the invention, the viscosity of the detergent compositions is controlled e.g., by the inclusion level of the inventive ether sulfates based on isomeric tridecyl alcohol mixtures and hence the inventive formulations do not compromise any organic solvent.

The compositions herein may further comprise from 30 wt % to 95 wt % of an aqueous liquid carrier, comprising water, in which the other essential and optional ingredients are dissolved, dispersed or suspended. More preferably the compositions of the present invention comprise from 45 wt % to 90 wt %, even more preferably from 60 wt % to 85 wt % of the aqueous liquid carrier. The aqueous liquid carrier, however, may contain other materials which are liquid, or which dissolve in the liquid carrier, at room temperature (25° C.) and which may also serve some other function besides that of an inert filler.

Compositions according to the invention may comprise at least one electrolyte.

Suitable electrolytes are preferably selected from inorganic salts, even more preferably selected from monovalent salts and divalent salts, most preferably sodium chloride and magnesium chloride.

The liquid manual dish wash compositions according to the invention optionally comprise from 0.1 wt % to 5 wt %, preferably from 0.2 wt % to 2 wt % of the composition of an electrolyte.

Manual dish wash formulations comprising the inventive ether sulfates based on isomeric tridecyl alcohol mixtures may also comprise at least one antimicrobial agent.

The antimicrobial agent may be selected from the list consisting of 2-phenoxyethanol (CAS-no. 122-99-6, for example Protectol® PE available from BASF) and 4,4′-dichloro-2-hydroxydiphenylether (CAS: 3380-30-1), and combinations thereof.

The 4,4′-dichloro-2-hydroxydiphenylether may be used as a solution, for example a solution of 30 wt % of 4,4′-dichloro-2-hydroxydiphenylether in 1,2-propyleneglycol, e.g. Tinosan® HP 100 available from BASF.

The inventive hand dish wash formulation may comprise at least one antimicrobial agent from the above list and/or a combination thereof, and/or a combination with at least one further antimicrobial agent not listed here.

The antimicrobial agent may be added to the inventive hand dish wash formulation in a concentration of 0.0001 wt % to 10 wt % relative to the total weight of composition. Preferably, the formulation contains 2-Phenoxyethanol in a concentration of 0.01 wt % to 5 wt %, more preferably 0.1 wt % to 2 wt % and/or 4,4′-dichloro 2-hydroxydiphenyl ether in a concentration of 0.001 wt % to 1 wt %, more preferably 0.002 wt % to 0.6 wt % (in all cases relative to the total weight of the composition including all components, water and solvents).

Compositions according to the invention may comprise at least one additional ingredient.

Examples of additional ingredients are such as but not limited to conditioning polymers, cleaning polymers, surface modifying polymers, soil flocculating polymers, rheology modifying polymers, enzymes, structurants, builders, chelating agents, cyclic diamines, structurants, emollients, humectants, skin rejuvenating actives, carboxylic acids, scrubbing particles, bleach and bleach activators, perfumes, malodor control agents, pigments, dyes, opacifiers, beads, pearlescent particles, microcapsules, antibacterial agents, pH adjusters including NaOH and alkanolamines such as monoethanolamines, and buffering agents.

The liquid manual dish wash detergent composition may have any suitable pH. Preferably the pH of the composition is adjusted to between 4 and 14. More preferably the composition has a pH of from 6 to 13, even more preferably from 6 to 10, most preferably from 7 to 9. The pH of the composition can be adjusted using pH modifying ingredients known in the art and is measured as a 10% product concentration in demineralised water at 25° C. For example, NaOH may be used and the actual wt % of NaOH may be varied and trimmed up to the desired pH such as pH 8.0. In one embodiment of the present invention, a pH >7 is adjusted by using amines, preferably alkanolamines, more preferably triethanolamine.

The compositions of the present invention preferably have a viscosity of from 50 to 10000 mPa*s, more preferably from 100 to 8000 mPa*s, and most preferably from 500 to 7000 mPa*s at 10 1/min and 20° C. (Brookfield).

Compositions according to the invention can be used for washing dishware. Said method of washing dishes comprises the step of applying the composition, preferably in liquid form, onto the dishware surface, either directly or by means of a cleaning implement, i.e., in neat form. The composition is applied directly onto the surface to be treated and/or onto a cleaning device or implement such as a dish cloth, a sponge or a dish brush without undergoing major dilution (immediately) prior to the application. The cleaning device or implement is preferably wet before or after the composition is delivered to it. In the method of the invention, the composition can also be applied in diluted form.

Both neat and dilute application give rise to superior cleaning performance, i.e., the formulations of the invention containing the inventive ether sulfates based on isomeric tridecyl alcohol mixtures exhibit excellent degreasing properties. The effort of removing fat and/or oily soils from the dishware is decreased due to the presence of the inventive ether sulfates based on isomeric tridecyl alcohol mixtures, even when the level of surfactant used is lower than in conventional compositions.

### (b) Laundry Formulations (Laundry Detergent Compositions)

Another aspect of the present invention is also a laundry formulation (i.e. laundry detergent composition), comprising at least one of the inventive ether sulfates based on isomeric tridecyl alcohol mixtures as described above.

The laundry detergent composition according to the invention can be liquid, gels, or solid compositions, solid embodiments encompassing, for example, powders and tablets. Liquid compositions are preferred, as mentioned above, and may be packaged as unit doses.

The inventive laundry detergent composition usually comprises at least one additional anionic surfactant.

Preferably the composition is formulated for increasing the foam level of a detergent composition, reducing the viscosity of a detergent composition and/or improving the degreasing properties of a detergent composition. Optional further benefits include improvement of soil removal.

Examples of suitable additional anionic surfactants are alkali metal and ammonium salts of C₁₂-C₁₈-fatty alcohol ether sulfates, of C₁₂-C₁₈-fatty alcohol polyether sulfates, of sulfuric acid half-esters of ethoxylated C₄-C₁₂-alkylphenols (ethoxylation: 3 to 50 mol of ethylene oxide/mol), of C₁₂-C₁₈-alkylsulfonic acids, of C₁₀-C₁₈-alkylarylsulfonic acids, preferably of n-C₁₀-C₁₈-alkylbenzene sulfonic acids (LAS), of C₁₀-C₁₈ alkyl alkoxy carboxylates and of soaps such as for example C₈-C₂₄-carboxylic acids. Preference is given to the alkali metal salts of the aforementioned ingredients, particularly preferably the sodium salts.

In a preferred embodiment of the present invention, additional anionic surfactants are selected from n-C₁₀-C₁₈-alkylbenzene sulfonic acids (LAS) and from fatty alcohol polyether sulfates, which, within the context of the present invention, are in particular sulfuric acid half-esters of ethoxylated C₁₂-C₁₈-alkanols (ethoxylation: 1 to 50 mol of ethylene oxide/mol), preferably of n-C₁₂-C₁₈-alkanols.

In one embodiment of the present invention, also alcohol polyether sulfates derived from other synthetic C₁₁-C₁₈-alkanols (ethoxylation: 1 to 50 mol of ethylene oxide/mol) may be employed.

Preferably, the alkoxylation group of both types of alkoxylated alkyl sulfates, based on C₁₂-C₁₈-fatty alcohols or based on other synthetic C₁₁-C₁₈-alcohols, is an ethoxylation group and an average ethoxylation degree of any of the alkoxylated alkyl sulfates is 1 to 5, preferably 1 to 3.

Preferably, the laundry detergent formulation of the present invention comprises from at least 30 wt % to 95 wt %, preferably in the range from greater than or equal to about 50 wt % to equal to or less than about 95 wt %, more preferably in the range from greater than or equal to 60 wt % to less than or equal to 95 wt %, and most preferably in the range from greater than or equal to 70 wt % to less than or equal to 90 wt % of one or more additional anionic surfactants, preferably selected from n-C₁₀-C₁₈-alkylbenzene sulfonic acids (LAS) and from fatty and/or synthetic alcohol polyether sulfates, as described above, relative to active matter comprising iso-tridecylalcohol-based ether sulfates, other ether sulfates and LAS, i.e. based on the particular overall composition but without other components, water and/or solvents.

Preferably, the laundry detergent formulation of the present invention contains from at least 1 wt % to 50 wt %, preferably in the range from greater than or equal to about 2 wt % to equal to or less than about 30 wt %, more preferably in the range from greater than or equal to 3 wt % to less than or equal to 25 wt %, and most preferably in the range from greater than or equal to 5 wt % to less than or equal to 25 wt % of anionic surfactants comprising ether sulfates based on isomeric tridecyl alcohol mixtures, other ether sulfates and LAS, based on the particular overall composition including other components, water and/or solvents.

In another embodiment of the present invention, the concentrated laundry detergent formulation of the present invention contains from at least 20 wt % to 50 wt %, preferably in the range from greater than or equal to about 25 wt % to equal to or less than about 45 wt %, more preferably in the range from greater than or equal to 25 wt % to less than or equal to 40 wt %, and most preferably in the range from greater than or equal to 30 wt % to less than or equal to 40 wt % of anionic surfactants comprising ether sulfates based on isomeric tridecyl alcohol mixtures, other ether sulfates and LAS, based on the particular overall composition including other components, water and/or solvents.

In a preferred embodiment of the present invention, the laundry detergent compositions based on the inventive ether sulfates based on isomeric tridecyl alcohol mixtures and the at least one other surfactant, preferably the at least one other anionic surfactant, can be obtained as clear, transparent and homogeneous liquid formulations at room temperature (25° C.), without any turbidity or precipitation of components.

In contrast, comparable products known in the art, specifically branched ether sulfates based on iso-tridecylalcohol prepared by oligomerization of propene and subsequent hydroformylation/hydrogenation, commercially available as Exxal 13 (Exxon Mobil), typically lead to cloudy, non-transparent and inhomogeneous liquid formulations at room temperature (25° C.), along the way often with precipitation of components, if combined with at least one other surfactant, preferably with at least one other anionic surfactant, more preferably if combined with LAS. Such non-transparent formulations are instable and thus technically not viable.

Compositions according to the invention may comprise at least one builder. In the context of the present invention, no distinction will be made between builders and such components elsewhere called “co-builders”. Examples of builders are complexing agents, hereinafter also referred to as complexing agents, ion exchange ingredients, and precipitating agents. Builders are selected from citrate, phosphates, silicates, carbonates, phosphonates, amino carboxylates and polycarboxylates.

Compositions according to the invention can comprise, for example, in the range from in total 0.1 to 70% by weight, preferably 10 to 50% by weight, preferably up to 20% by weight, of builder(s), especially in the case of solid formulations. Liquid formulations according to the invention preferably comprise in the range of from 0.1 to 8% by weight of builder.

Formulations according to the invention can comprise one or more alkali carriers. Alkali carriers ensure, for example, a pH of at least 9 if an alkaline pH is desired. Of suitability are, for example, the alkali metal carbonates, the alkali metal hydrogen carbonates, and alkali metal metasilicates mentioned above, and, additionally, alkali metal hydroxides. A preferred alkali metal is in each case potassium, particular preference being given to sodium. In one embodiment of the present invention, a pH >7 is adjusted by using amines, preferably alkanolamines, more preferably triethanolamine.

In one embodiment of the present invention, the laundry formulation according to the invention comprises additionally at least one enzyme.

Useful enzymes are, for example, one or more lipases, hydrolases, amylases, proteases, cellulases, hemicellulases, phospholipases, esterases, pectinases, lactases and peroxidases, and combinations of at least two of the foregoing types.

Enzyme can be incorporated at levels sufficient to provide an effective amount for cleaning. The preferred amount is in the range from 0.001% to 5% of active enzyme by weight in the detergent composition according to the invention. Together with enzymes also enzyme stabilizing systems may be used such as for example calcium ions, boric acid, boronic acid, propylene glycol and short chain carboxylic acids. In the context of the present invention, short chain carboxylic acids are selected from monocarboxylic acids with 1 to 3 carbon atoms per molecule and from dicarboxylic acids with 2 to 6 carbon atoms per molecule. Preferred examples are formic acid, acetic acid, propionic acid, oxalic acid, succinic acid, HOOC(CH₂)₃COOH, adipic acid and mixtures from at least two of the foregoing, as well as the respective sodium and potassium salts.

Compositions according to the invention may comprise one or more bleaching agent (bleaches).

Preferred bleaches are selected from sodium perborate, anhydrous or, for example, as the monohydrate or as the tetrahydrate or so-called dihydrate, sodium percarbonate, anhydrous or, for example, as the monohydrate, and sodium persulfate, where the term “persulfate” in each case includes the salt of the peracid H₂SO₅ and also the peroxodisulfate.

Formulations according to the invention can comprise one or more bleach catalysts. Bleach catalysts can be selected from oxaziridinium-based bleach catalysts, bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and also cobalt-, iron-, copper- and ruthenium-amine complexes can also be used as bleach catalysts.

Formulations according to the invention can comprise one or more bleach activators, for example tetraacetyl ethylene diamine, tetraacetylmethylene diamine, tetraacetylglycoluril, tetraacetylhexylene diamine, acylated phenolsulfonates such as for example n-nonanoyl- or isononanoyloxybenzene sulfonates, N-methylmorpholinium-acetonitrile salts (“MMA salts”), trimethylammonium acetonitrile salts, N-acylimides such as, for example, N-nonanoylsucci-nimide, 1,5-diacetyl-2,2-dioxohexahydro-1,3,5-triazine (“DADHT”) or nitrile quats (trimethylammonium acetonitrile salts).

Formulations according to the invention can comprise one or more corrosion inhibitors. In the present case, this is to be understood as including those ingredients which inhibit the corrosion of metal. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxyhydroquinone, gallic acid, phloroglucinol or pyrogallol.

In one embodiment of the present invention, formulations according to the invention comprise in total in the range from 0.1 to 1.5% by weight of corrosion inhibitor.

Formulations according to the invention may also comprise further cleaning polymers and/or soil release polymers.

The additional cleaning polymers may include, without limitation, multifunctional polyethylene imines (for example BASF's Sokalan® HP20) and/or multifunctional diamines (for example BASF's Sokalan® HP96).

Laundry detergent compositions comprising the inventive ether sulfates may also comprise at least one antimicrobial agent.

The antimicrobial agent may be selected from the list consisting of 2-phenoxyethanol (CAS-no. 122-99-6, for example Protectol® PE available from BASF) and 4,4′-dichloro-2-hydroxydiphenylether (CAS: 3380-30-1), and combinations thereof.

The 4,4′-dichloro-2-hydroxydiphenylether may be used as a solution, for example a solution of 30 wt % of 4,4′-dichloro-2-hydroxydiphenylether in 1,2-propyleneglycol, e. g. Tinosan® HP 100 available from BASF.

Formulations according to the invention may comprise at least one additional surfactant, selected from anionic, non-ionic, amphoteric and from zwitterionic surfactants.

Examples of (additional) anionic surfactants are alkali metal and ammonium salts of C₈-C₁₆-alkyl sulfates and of C₁₂-C₁₈ sulfo fatty acid alkyl esters, for example of C₁₂-C₁₈ sulfo fatty acid methyl esters. Preferred non-ionic surfactants are alkoxylated alcohols and alkoxylated fatty alcohols, di- and multiblock copolymers of ethylene oxide and propylene oxide and reaction products of sorbitan with ethylene oxide or propylene oxide, furthermore alkylphenol ethoxylates, alkyl glycosides, polyhydroxy fatty acid amides (glucamides).

Further suitable non-ionic surfactants are selected from di- and multiblock copolymers, composed of ethylene oxide and propylene oxide. Further suitable non-ionic surfactants are selected from ethoxylated or propoxylated sorbitan esters. Alkylphenol ethoxylates or alkyl polyglycosides or polyhydroxy fatty acid amides (glucamides) are likewise suitable. An overview of suitable further non-ionic surfactants can be found in EP-A 0 851 023 and in DE-A 198 19 187.

Mixtures of two or more different non-ionic surfactants may also be present.

Suitable amphoteric surfactants are so-called amine oxides, such as lauryl dimethyl amine oxide (“lauramine oxide”).

Examples of zwitterionic surfactants are C₁₂-C₁₈-alkylbetaines and sulfobetaines.

Detergent compositions according to the invention may also comprise water and additional organic solvents, e.g. ethanol or propylene glycol.

Further optional ingredients may be but are not limited to viscosity modifiers, cationic surfactants, foam boosting or foam reducing agents, perfumes, dyes, optical brighteners, and dye transfer inhibiting agents.

### EXAMPLES

In the following paragraphs, several experimental examples are given in order to illustrate some aspects of the present invention.

### Synthesis Example

Synthesis of the inventive ether sulfates based on an isomeric tridecyl alcohol mixture:
i) The isomeric tridecyl alcohol mixture used for preparing the inventive ether sulfates based on an isomeric tridecyl alcohol mixture has been manufactured as described in U.S. Pat. No. 6,963,014 B1. The alcohol has been obtained with a mean degree of branching of ca. 2.3.
ii) Ethoxylation of the isomeric tridecyl alcohol mixture has been performed in accordance with the disclosures of U.S. Pat. No. 6,963,014 B1. An average amount of 3 EO per OH group has been added to the branched iso-tridecylalcohol from step (i).
iii) Sulfation of the ethoxylated isomeric tridecyl alcohol mixture from step (ii):

The temperature of the alcohol ethoxylate, an ethoxylated isomeric tridecyl alcohol mixture (isomeric compounds all having an identical number of thirteen carbon atoms in the alkyl rest, i.e., ethoxylates based on “pure” iso-tridecylalcohols) with a mean degree of branching of ca. 2.3, ethoxylated with 3 EO per OH group on average (reaction product from step (ii)), in the stirred storage vessel was kept at 50-60° C. The sulfation of the alcohol ethoxylate was performed on a state of art falling film sulfonation reactor at a reactor temperature of 35° C. with a molar ratio of SO₃/alcohol ethoxylate=1.0-1.05 with dry air/SO₃ containing 5 vol % of SO₃. The product was neutralized after degassing in a neutralization loop with a mixture of caustic soda (50%) and water, calculated to obtain a concentration of approx. 70% active at a temperature of 65° C., keeping the pH-value in the range of 9-12 to avoid hydrolysis of the product.

The resulting inventive ether sulfates based on an isomeric tridecyl alcohol mixture were bleached subsequently in a stirred vessel by applying 0.3% hydrogen peroxide as 35% solution at 50-80° C. The final pH was adjusted by adding caustic soda to a pH value of 9.9.

The final product was obtained as a paste with 70.6 wt % active matter content and a degree of sulfation of 97%.

### Application Examples 26

Several hand dish wash (HDW) formulations were prepared, containing the inventive ether sulfates based on an isomeric tridecyl alcohol mixture in different amounts, and several HDW formulations without the inventive compound.

The formulations were prepared by mixing the surfactants as aqueous solutions, and then the pH was adjusted with base.

For LAS (Linear alkyl benzene sulfonic acid) based formulations, neutralization was done in the beginning with NaOH, then the rest of ingredients was added in any order, followed by pH adjustment at the end.

The composition of the inventive compositions and the comparative compositions, as well as the results of different application tests, are given in table 1 below.

TABLE 1
Composition of HDW formulations and application test results

| % AM | F.1 | F.2 | F.3 | F.4 | F.5 | F.6 | F.7 | F.8 |
|---|---|---|---|---|---|---|---|---|
| FAEOS*¹ | 16 | — | 10 | 10 | 5 | — | 9 | 3.937 |
| Iso-C₁₃ EOS*² | — | 16 | 6 | — | 5 | 10 | — | 3.937 |
| LAS*³ | — | — | — | — | — | — | 9 | 7.875 |
| Dehyton ® PK45 | — | — | — | — | — | — | — | — |
| (Cocamidopropyl | | | | | | | | |
| betaine) | | | | | | | | |
| Dehyton ® PL | | | | | | | 2 | 1.75 |
| (Lauryl | | | | | | | | |
| amineoxide) | | | | | | | | |
| Dehyton ® PK45/PL | — | — | — | 6 | 6 | 6 | — | — |
| (3:1 Mixture) | | | | | | | | |
| pH-Value | 7.5 | 7.5 | 7.5 | 7.5 | 7.5 | 7.5 | 7.5 | 7.5 |
| Appearance at | clear, | clear, | clear, | clear, | clear, | clear, | clear, | clear, |
| 25° C. | transparent, | transparent, | transparent, | transparent, | transparent, | transparent, | transparent, | transparent, |
| | homo- | homo- | homo- | homo- | homo- | homo- | homo- | homo- |
| | geneous | geneous | geneous | geneous | geneous | geneous | geneous | geneous |
| Foam Height | 12.5 | 36.5 | 27.5 | — | — | — | — | — |
| (Cylinder Test) | | | | | | | | |
| [mL] | | | | | | | | |
| Numbers of plates | 19 | 26 | 24 | 39 | 42 | 46 | — | — |
| washed; IKW soil | | | | | | | | |
| Numbers of plates | — | — | — | — | — | — | 12 | 13 |
| washed; Beef | | | | | | | | |
| Tallow soil | | | | | | | | |
| Viscosity | — | — | — | 16720 | 6520 | 6360 | — | — |
| Brookfield (Spindle | | | | | | | | |
| 5; 10 RPM) | | | | | | | | |
| [mPa*s; 20° C.] | | | | | | | | |
| Emulsification | — | — | — | 19 | 16 | 11 | 30 | 28 |
| Test: Stability olive | | | | | | | | |
| oil (1 hour) [mL | | | | | | | | |
| surfactantsolution] | | | | | | | | |

*¹= C₁₂-C₁₄ linear alkyl ether sulfate, sodium salt
*²= inventive ether sulfates based on an isomeric tridecyl alcohol mixturee, sodium salt (prepared according to the description above)
*³= Linear alkyl benzene sulfonic acid, sodium salt
F.2, F.3, F.5, F.6 and F.8 are inventive formulations, containing the inventive ether sulfates based on an isomeric tridecyl alcohol mixture.
F.1, F.4 and F.7 are comparative formulations.

### Foam Test in Cylinders

### Test Procedure:

70 mL of a dishwashing detergent solution, prepared by dissolution of 2 g of the respective HDW formulation in 1 L of hard water (14° dH), have been transferred into 250 mL glass cylinders. To each of the cylinders, 0.5 g of rapeseed oil (as a model soil for hand dish application) has been added. Then, the cylinders have been agitated manually, by shaking them vertically (5 times up and down). Subsequently, the solutions were stored without agitation at room temperature and evaluated after 2 minutes in terms of their foam height (total foam height minus the amount of remaining liquid solution).

### Test Results:

It can be clearly seen from the results in table 1, that inventive formulation F.2 exhibits significantly more foam (ca. 30%) than comparative formulation F.1, which is based solely on linear alkyl ether sulfate.

### Determination of Number of Plates Washed

The number of plates washed was determined according to the IKW test method, using a soil composition according to the original IKW protocol (IKW soil) or using beef tallow as soil component (beef tallow soil), respectively, for some compositions.

The original IKW recommendation is a manual test method, however, in the context of the present invention, all tests have been performed by using the semi-automatic version of the test protocol.

Background: The recommendation for the quality of the cleaning performance of manual dishwashing detergents was elaborated in an IKW Working Group (www.ikw.org) by ColgatePalmolive GmbH, Dalli Werke Wäsche- und Körperpflege GmbH & Co. KG, fit GmbH, Henkel KGaA, Luhns GmbH, Reckitt Benckiser plc., Werner & Mertz GmbH, and published by The German Cosmetic, Toiletry, Perfumery and Detergent Association e.V. (IKW) in 2002.

### Test Procedure:

First, the dishwashing detergent solution, prepared by dissolution of 4 g of the respective HDW formulation in 5 L of water (=concentration of 0.8 g/L), has been foamed up, using a pump. Water with a hardness of 16° dH has been employed. The soak temperature in the pot at the beginning of the test was 45±1° C. Then, soiled plates (dosage of soil per plate: 5 g) have been washed semi-automatically, using a semi-automatic dish washing device (brush, attached to a mechanical stirrer, electrically operated). At the beginning of the washing-up process, the foam permanently covered the water surface. When the foam did not cover the washing liquor anymore (=compete collapse of the foam layer), the test has been stopped and the number of plates has been recorded.

Due to the specific endpoint detection of the plates wash test, this method is perfectly suitable to determine the foam properties of a formulation and its components: The higher the number of plates that can be washed, the higher the amount of foam generated during the washing process and/or the higher the stability of the foam.

The test error of this method is plus/minus one plate.

### Test Results:

It can be clearly seen from the results in table 1, that inventive formulations F.2 and F.3 exhibit higher number of plates (i.e., higher and/or more stable foam) than comparative formulation F.1, based solely on linear alkyl ether sulfate. The same results are observed if formulations containing additional co-surfactants have been tested: Formulations F.5 and F.6 exhibit higher number of plates than comparative formulation F.4. In case of LAS-containing formulations (F.7-F.8) it can be seen by comparing comparative formulation F.7 and inventive formulation F.8, that even a decrease of the overall surfactant concentration (F.7: 20 wt % AM; F.8: 17.5 wt % AM), which typically leads to lower foam levels if the type and ratio of the ingredients is not changed, will not lead to a lower number of plates if at the same time half of the (reduced) amount of the linear alkyl ether sulfate will be replaced by the inventive ether sulfates based on an isomeric tridecyl alcohol mixture. In contrast, it will even lead to a slight increase of the number of plates (and thus foam levels), demonstrating the capability of the inventive ingredient to contribute to high(er) foam levels at lower AM concentrations, thus leading to more weight-efficient detergent formulations.

### Method to Determine Viscosity

### Test Procedure:

The viscosity of some of the HDW formulations was determined according to EN12092 (dating from Feb. 1, 2002), using a Brookfield viscometer (spindle 5) at 10 rpm.

### Test Results:

It can be clearly seen from the results in table 1, that inventive formulations F.5 and F.6 exhibit significantly lower viscosities than comparative formulation F.4 (reduction of more than 60%), at identical AM (active matter content), all 16 wt % AM). The incorporation of the inventive ether sulfates based on an isomeric tridecyl alcohol mixture leads to detergents with lower viscosities which are much easier to handle (in production, for end-consumers, etc.). The use of unwanted other technologies to reduce the viscosity, like addition of alcohols (ethanol, propylene glycol), which add additional cost to the formulations and might impact their performance profile, can thus be avoided.

### Emulsification Stability Test Method:

The method was used to evaluate the emulsifier potential of surfactants with fats and oils. The emulsifier potential of a compound is linked to its degreasing properties in hand dish wash application.

### Test Procedure:

50 mL of oil and 50 mL of surfactant solution (2% AM in water) were mixed thoroughly (1200 rpm for 2 minutes) with a mechanical stirrer. Then, the emulsions (each: 100 ml total volume) were stored without agitation at room temperature and evaluated after 1 hour in terms of their stability (determination of separated surfactant solution). The lower the remaining level of surfactant solution (detected as volume, in mL; cf. table 1) means the better the emulsion stability and emulsification properties of the test solution.

### Test Results:

It can be clearly seen from the results in table 1 that inventive formulations F.5 and F.6 exhibit significantly lower levels of separated surfactant solution than comparative formulation F.4. Thus, the stability of the emulsions containing the inventive ether sulfates based on an isomeric tridecyl alcohol mixture is significantly improved and an improved degreasing property in hand dish application can be expected. In case of LAS-containing formulations (F.7-F.8) it can be seen by comparing comparative formulation F.7 and inventive formulation F.8, that again a decrease of the overall surfactant concentration (F.7: 20 wt % active matter, AM; F.8: 17.5 wt % AM), which typically leads to decreased emulsifying properties of a formulation if the type and ratio of the ingredients is not changed, will not lead to a significantly changed amount of separated surfactant solution if at the same time half of the (reduced) amount of the linear alkyl ether sulfate will be replaced by the inventive ether sulfates based on an isomeric tridecyl alcohol mixture. More specifically, the emulsion stability after 1 hour is even slightly improved, demonstrating the capability of the inventive ingredient to contribute to better emulsifying properties and potentially also to better degreasing properties at lower AM concentrations, thus leading to more weight-efficient detergent formulations.

### Summary Application Tests:

The experiments show the advantages of using the inventive compound in detergent compositions. For example, an increase in foam creation can be achieved, and at the same time the viscosity is decreased. In addition, the emulsifying properties and potentially also the degreasing properties can be improved, even in case of overall lower surfactant levels.

### Experimental Data Regarding Biodegradability

An (i) inventive ether sulfate based on an isomeric tridecyl alcohol mixture and a (ii) comparative ether sulfate were subjected to a controlled biodegradation for 28 days, according to OECD norm 301B.
(i) The inventive ether sulfate based on an isomeric tridecyl alcohol mixture as prepared according to synthetic procedure described above is based on a purely C13 iso-tridecylalcohol with a degree of branching of ca. 2.3 (determined as described above), ethoxylated with three ethylene oxide units on average, and sulfated to a degree of approximately 97%.
(ii) The comparative ether sulfate (prepared by ethoxylation of an iso-tridecylalcohol-containing alcohol mixture (Exxal 13 from Exxon Mobil) with 3 EO per OH group on average and subsequently sulfated, according to the identical procedures as described above in step (i) and (ii) for the inventive example) is based on a mixture containing an iso-tridecylalcohol with a degree of branching of ca. 3.1 (determined as described above), ethoxylated with three ethylene oxide units on average, and sulfated to a degree of approximately 97%.

Results: The (i) inventive ether sulfate showed a biodegradability of 62% (according to OECD norm 301B), whereas the (ii) comparative ether sulfate showed a biodegradability (according to OECD norm 301B) of only 45%. Thus, the inventive ether sulfate, due to its considerably higher biodegradability, is better suited for use in Home Care applications (e.g., laundry detergents or dishwashing).

1. A process of manufacturing ether sulfates based on isomeric tridecyl alcohol mixtures, comprising the steps of
a. bringing a butene-carrying C4-hydrocarbon stream comprising less than 5% by weight, based on the butene fraction, of isobutene into contact with a nickel-containing heterogeneous catalyst at elevated temperature,
b. isolating a C12-olefin fraction from the reaction mixture,
c. hydroformylating the C12-olefin fraction by reaction with carbon monoxide and hydrogen in the presence of a cobalt catalyst,
d. hydrogenating the product from c), wherein the isomeric tridecyl alcohol mixture obtained after steps a) to d) above has a mean degree of branching in a range of from 2.1 to 2.5,
e. ethoxylating the product from d), wherein 1 to 5 EO units per OH group are used on average,
f. sulfating the product from e) with SO₃, and
g. neutralizing the product from f) with an aqueous solution of a base.

2. Ether sulfates based on isomeric tridecyl alcohol mixtures, obtained by a process according to claim 1.

3. The process of manufacturing ether sulfates according to claim 1, wherein 2 to 4 EO units per OH group are used on average in step e).

4. The process of manufacturing ether sulfates according to claim 1, wherein the sulfation in step f) is done in a falling film sulfonation reactor.

5. The process of manufacturing ether sulfates according to claim 1, wherein the degree of sulfation in step f) is in a range of from 80% to 100%.

6. The process of manufacturing ether sulfates according to claim 1, wherein the neutralization in step g) is performed with an aqueous solution of a base selected from the list consisting of NaOH, KOH, triethanolamine, and ammonia.

7. The process of manufacturing ether sulfates according to claim 1, wherein the isomeric tridecyl alcohol mixture obtained after steps a) to d) above has a mean degree of branching in a range of from 2.2 to 2.4, wherein the mean degree of branching is the statistical mean of the degree of branching of the molecules of a sample, wherein the mean number of methyl groups in the molecules of a sample may be determined by ¹H-NMR spectroscopy.

8. The ether sulfates based on isomeric tridecyl alcohol mixtures according to claim 2, having a biodegradability according to OECD norm 301B of at least 60%.

9. A composition comprising ether sulfates based on isomeric tridecyl alcohol mixtures according to claim 2, and water.

10. The composition according to claim 9, comprising 5 to 99% by weight water.

11. (canceled)

12. The composition preferably liquid composition, according to claim 9, comprising additionally at least one anionic surfactant, selected from the group consisting of other ether sulfates, alkyl sulfates and linear alkylbenzene sulfonate (LAS).

13. The composition according to claim 9, further comprising at least one co-surfactant.

14. The composition according to claim 9, which does not comprise any cationic surfactants.

15. The composition according to claim 9, which does not comprise any non-ionic surfactants based on C₁₂ to C₂₀ alcohol alkoxylates.

16. The composition according to claim 12, comprising up to 70 wt %, ether sulfates-based on isomeric tridecyl alcohol mixtures, relative to active matter comprising ether sulfates based on isomeric tridecyl alcohol mixtures, other anionic surfactants and co-surfactants.

17. The composition according to claim 12, comprising 30 to 95 wt % of an additional anionic surfactant, selected from the group consisting of other ether sulfates, alkyl sulfates and linear alkylbenzene sulfonate (LAS), relative to active matter comprising ether sulfates based on isomeric tridecyl alcohol mixtures, other ether sulfates, alkyl sulfates and linear alkylbenzene sulfonate (LAS).

18. The composition according to claim 9, wherein the composition is a detergent composition.

19. (canceled)

20. A method of using ether sulfates based on isomeric tridecyl alcohol mixtures according to claim 2, the method comprising using the ether sulfates for increasing the foam level in a detergent composition, reducing the viscosity of a detergent composition and/or improving the degreasing properties of a detergent composition.

21. The process of manufacturing ether sulfates according to claim 1, wherein neutralizing the product from f) comprises neutralizing the product from f) with an aqueous solution of a base selected from the group consisting of alkaline metals, earth alkaline metals, and amines.

22. The process of manufacturing ether sulfates according to claim 1, wherein 2.5 to 3.5 EO units per OH group are used on average in step e).
